(19) United States
(12) Patent Application Publication
(10) Pub. No.: US 2021/0275431 A1
(43) Pub. Date: Sep. 9, 2021

(54) SKIN FORMULATION

(71) Applicant: SWISS SKIN POWER AG, CH-8001 ZÜRICH (CH)

(72) Inventors: Daniel FUCHS, Zürich (CH); Neda HEDAYAT, Zürich (CH)

(73) Assignee: SWISS SKIN POWER AG, CH-8001 ZÜRICH (CH)

(21) Appl. No.: 17/252,608

(22) PCT Filed: Jun. 20, 2019
(86) PCT No.: PCT/EP2019/066347

§ 371 Date: Dec. 15, 2020

(30) Foreign Application Priority Data
Jun. 20, 2018 (CH) 00784/18

(51) Int. Cl. A61K 8/88; A61Q 19/08; A61Q 19/00; A61K 8/9789

(52) U.S. Cl. CPC A61K 8/88; A61Q 19/08; A61K 2800/522; A61K 8/9789; A61Q 19/007

(57) ABSTRACT

A cosmetic composition includes at least one gamma-PGA composition, at least one plant stem cell extract, at least one additional peptide composition and at least one vitamin composition.

### BACKGROUND OF THE INVENTION

### Field of the Invention

The invention lies in the field of skin care cosmetic products. More particularly, it is related to a cosmetic composition based on gamma-PGA or an acceptable salt thereof, further comprising one or more of a plant stem cell extract, a peptide composition and a vitamin composition, as well as the use of such a cosmetic composition in a cosmetic method.

### Discussion of Related Art

As the human skin is a very sensitive organ, a wide variety of cosmetic products has been developed in order to prevent its deterioration such as premature aging of the skin, age spots and the loss of firmness. Furthermore, many people suffer from dry skin caused by low humidity, which is especially problematic in winter, as low temperature and dry air further dehydrate the skin. Therefore, various different moisturizers that aid to keep water in the skin and humectants that aid to draw water from the air have been developed and included in a variety of skin care products such as serums, foams, crèmes, ointments, gels, etc.

However, the requirements for effective and suitable cosmetic skin care products are high. Firstly, these products should possess high water retention capacity and decrease water loss of the human skin. Secondly, the ingredients should not react with themselves, i.e. they should not cancel their effects. Thirdly, skin care products should not result in an uncomfortable oily or sticky feeling of the skin.

There are many different organic moisturizers being used in a variety of cosmetic and toiletry products on the market. A very prominent skin care agent is hyaluronic acid (HA), which naturally occurs in the basal layer of the epidermis. Due to its ability to store a relatively large amount of water per gram substance and due to its involvement in several skin healing processes, such as repair of UV damages, it is commonly employed in the beauty and cosmetic industry. In the human body, HA can easily be degraded by a family of enzymes called hyaluronidases. Cosmetic products containing HA can therefore only act as short term moisturizers, as the HA is readily degraded by these enzymes. Furthermore, HA as well as many other ingredients of skin care products, such as various peptides, cannot enter the human skin due to their relatively large molecular size and thus show some limitations. Another disadvantage of HA is the demanding manufacturing either from animal sources or from Streptococcus bacteria, which results in a high price and limited availability. Consequently, skin care products which may aid to preserve the human HA or which can aid to decrease the required amount of HA in a skin care product are highly desirable.

Due to its strong moisturizing ability, gamma-polyglutamic acid (gamma-PGA) has also been integrated into skin-care products. This compound is able to strengthen the moisturizing ability of the skin and thus prevents the skin from drying out. Due to its molecular structure, gamma-PGA can absorb and retain skin moisture efficiently and can form a silky film on the surface of the skin. Furthermore, gamma-PGA can increase and maintain the content of HA, which results in an increased moisturizing effect.

As many skin care products show unsatisfactory results with respect to wrinkle prevention and/or smoothing, improved skin care products are still required. Furthermore, it would be preferable to provide products, which concomitantly can protect and preserve the human skin stem cells in order to maintain and optionally increase the self-healing properties of the skin.

### SUMMARY OF THE INVENTION

It is an overall objective of the present invention to improve the state of the art regarding cosmetic compositions and use of such compositions, thereby preferably avoiding or overcoming the disadvantages of the prior art fully or partly.

In specific embodiments, a cosmetic composition according to the invention can retain moisture for a long time, raises the skin elasticity and/or increases the natural moisturizing factor. Furthermore, the specific embodiments may aid to preserve the human HA. The overall objective is in a general way achieved by the subject of the independent claims. Further advantageous and exemplary embodiments follow from the dependent claims, the description and the figures.

According to a first aspect, the invention is directed towards a cosmetic composition comprising (i) at least one gamma-PGA composition, (ii) at least one plant stem cell extract, (iii) at least one additional peptide composition and (iv) at least one vitamin composition.

According to a further aspect, the invention is directed towards a cosmetic method for increasing the natural moisturizing factor and/or for the prevention of premature skin degradation and/or for the prevention of age spots. The method comprises the step of topically applying a composition according to the invention.

According to a preferred embodiment, the gamma-PGA composition comprises gamma-PGA and/or an acceptable salt thereof, preferably potassium hydrolyzed polygamma-glutamate, which is the hydrolysate of potassium polygamma-glutamate derived by acid, enzyme or other method of hydrolysis.

In a further embodiment, the gamma-PGA composition comprises an amount of low-molecular weight gamma-PGA or acceptable salt thereof with a molecular weight between 300 and 1500 Da, preferably between 800 and 1200 Da. Optionally, the gamma-PGA composition may comprise an amount of high-molecular weight gamma-PGA or acceptable salt thereof with a molecular weight between 1 MDa and 5 MDa, preferably between 2 MDa and 3 MDa.

In a preferred embodiment, the at least one stem cell extract is selected from one or more of apple stem cell extract, preferably Malus domestics stem cell extract; argan stem cell extract, preferably Argania spinosa stem cell extract and/or alp rose stem cell extract, preferably Rhododendron ferrugineum stem cell extract.

In another embodiment the additional peptide composition comprises one or more additional peptides selected from acetyl hexapeptide-8, palmitoyl pentapeptide-4, palmitoyl tripeptide-1, palmitoyl tetrapeptide-7, dipeptide-2, hesperidin methyl chalcone and/or dipeptide diaminobutyroyl benzylamide diacetate.

In a further embodiment, the vitamin composition comprises vitamin C, vitamin E and/or vitamin A or a suitable derivative of these vitamins.

In a further embodiment, the amount of the gamma-PGA composition of the cosmetic composition is between 0.05 and 4 wt/wt %, preferably between 0.1 and 1 wt/wt %.

In a further embodiment, the amount of plant stem cell extracts of the cosmetic composition is between 0.003 and 0.015 wt/wt %, preferably between 0.005 and 0.01 wt/wt %; the amount of the additional peptide composition is between 1·10⁻⁵ and 1·10⁻⁴ wt/wt %, preferably between 5·10⁻⁵ and 9·10⁻⁵ wt/wt %; and the amount of the vitamin composition is between 0.01 and 0.1 wt/wt %, preferably between 0.04 and 0.08 wt/wt %.

A preferred embodiment of the invention is a cosmetic composition wherein the gamma-PGA composition comprises potassium hydrolyzed polygamma-glutamate. The cosmetic composition further comprises apple stem cell extracts, argan stem cell extracts and alp rose stem cell extracts. The additional peptide composition comprises acetyl hexapeptide-8, palmitoyl pentapeptide-4, palmitoyl tripeptide-1 and palmitoyl tetrapeptide-7. In addition, in such an embodiment, the vitamin composition comprises ascorbyl glucoside and tocopheryl acetate.

In a further embodiment, the cosmetic composition may further comprise a UV protection agent, i.e. an UV absorbing agent such as titanium dioxide or an organic UV absorbing agent.

In another embodiment, the cosmetic composition may comprise hyaluronic acid or a suitable salt thereof, such as sodium or potassium hyaluronate. Preferably, the hyaluronic acid or the suitable salt thereof is present in an amount of between 0.005 and 0.03 wt/wt %.

In a further embodiment, the cosmetic composition may also comprise superoxide dismutase, typically in an amount between 0.001 and 0.01 wt/wt %. In a further embodiment, the cosmetic composition may comprise glycine soy protein, preferably in an amount of 0.001 to 0.01 wt/wt %.

In a preferred embodiment of the invention, the cosmetic composition may be in the form of a serum, a crème or a foam.

### BRIEF DESCRIPTION OF SEVERAL VIEWS OF THE DRAWINGS

FIG. 1 Comparative absorbent effect: hyaluronic acid (♦), other PGA (▪) and composition of the invention (▴) (powder at 25° C., 81% relative humidity);

FIG. 2A is a chart showing a comparative moisture retention effect of hyaluronic acid (♦), other PGA ( [symbol] ) and composition of the invention (▴) (1% solution at 25° C., 18% relative humidity);

FIG. 2B is a chart showing a comparative moisture retention effect of hyaluronic acid (♦), other PGA ( [symbol] ) and composition of the invention (▴);

FIG. 3A is a chart showing Skin elasticity;

FIG. 3B is a chart showing moisture content; and

FIG. 3C is a chart showing transepidermal water loss (TEWL): HA 2000 KDa (▪), Collagen 300 KDa ( [symbol] ), PGA 1000 KDa ( [symbol] ), composition of the invention (□).

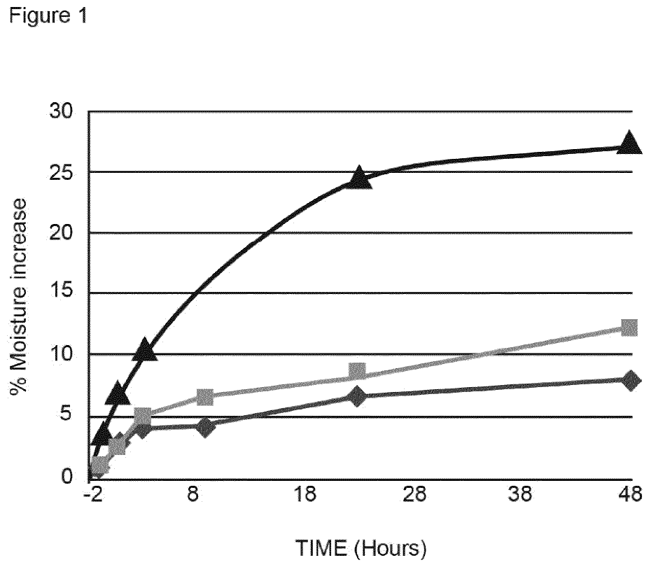

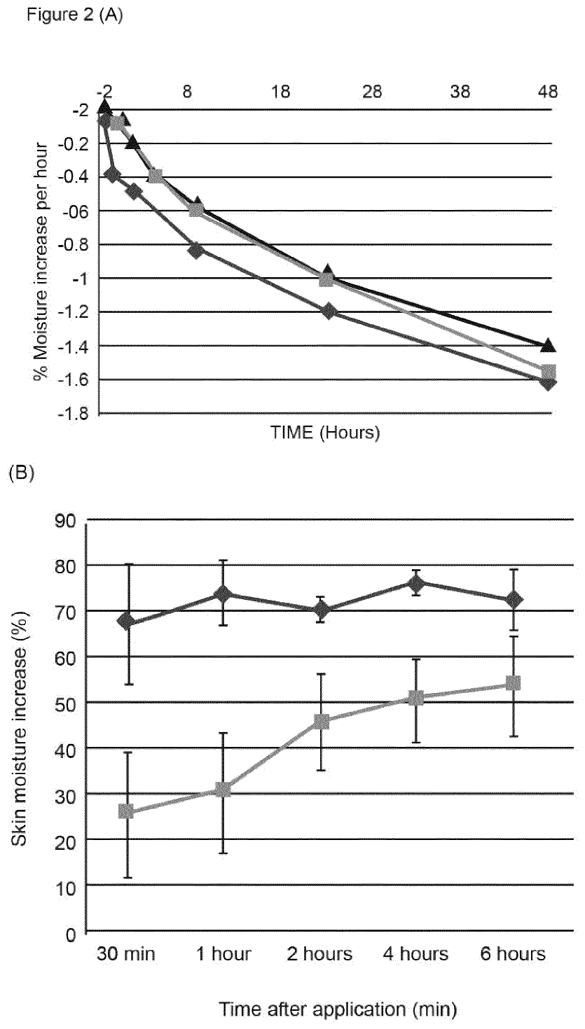

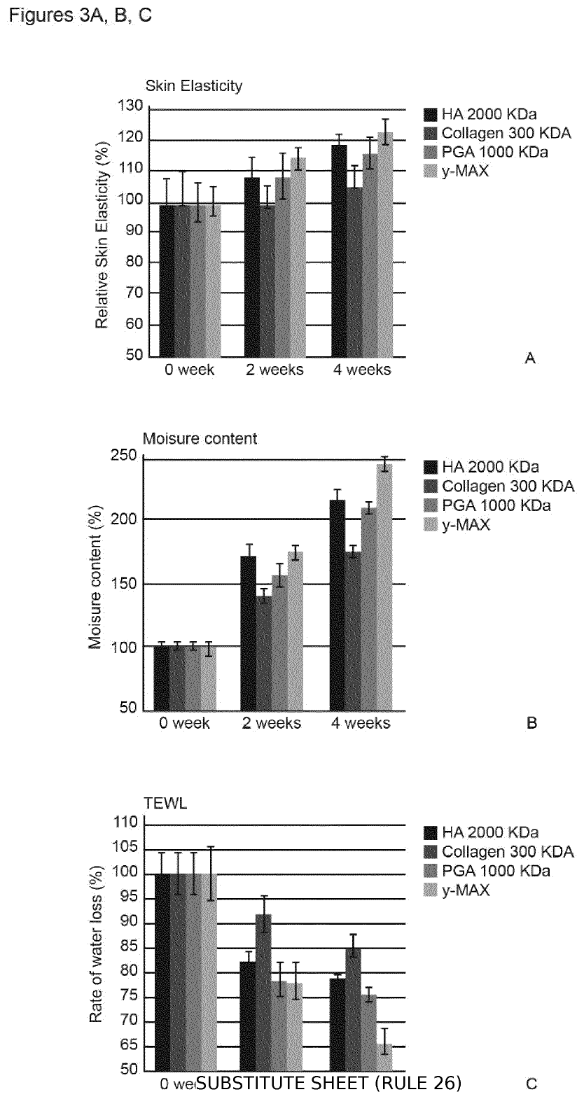

### DETAILED DESCRIPTION OF THE INVENTION

According to a first aspect, the invention is directed towards a cosmetic composition comprising (i) at least one gamma-PGA composition, (ii) at least one plant stem cell extract, (iii) at least one additional peptide composition and (iv) at least one vitamin composition. This cosmetic composition according to the invention provides an excellent moisturizer by providing a combination of at least one gamma-PGA composition, at least one plant stem cell extract, at least one additional peptide composition and at least one vitamin composition. The vitamin composition may comprise at least one vitamin, which is beneficial for the human skin.

As used herein, a gamma-PGA composition can comprise at least one gamma-PGA, but can also comprise a plurality of gamma-PGAs with different molecular weights from 100 Da to 5 MDa. Consequently, the term gamma-PGA can comprise gamma-PGA molecules with the same or with different molecular weights. Furthermore, a gamma-PGA composition can comprise gamma-PGA salts, such as sodium, potassium, magnesium, calcium, ammonium salts or the like. In accordance to the statement above, a gamma-PGA salt can comprise gamma-PGA salt molecules with the same or with different molecular weights. In a specific embodiment, the gamma-PGA composition comprises an amount of low-molecular weight gamma-PGA or acceptable salt thereof with a molecular weight between 300 and 1500 Da, preferably between 800 and 1200 Da. Optionally, the gamma-PGA composition may comprise an amount of high-molecular weight gamma-PGA or acceptable salt thereof with a molecular weight between 1 MDa and 5 MDa, preferably between 2 MDa and 3 MDa. In a further embodiment, the amount of the gamma-PGA composition of the cosmetic composition is between 0.05 and 4 wt/wt %, preferably between 0.1 and 1 wt/wt %.

Plant stem cell extracts are known to the person skilled in the art. These can for example be obtained by mechanical damage of a plant, upon which a callus forms that can be transferred to a nutrient medium to obtain cell lines. These can then further be grown in bioreactors upon monitoring physical and chemical parameters. Then the components within the cells are released by suitable mechanical or chemical means such as pressurization or treatment with a suitable reagent. Specifically, plant stem cell extracts include preferably apple stem cell extracts. Thus, in a preferred embodiment, the at least one stem cell extract is selected from one or more of apple stem cell extract, preferably Malus domestica stem cell extract; argan stem cell extract, preferably Argania spinosa stem cell extract and/or alp rose stem cell extract, preferably Rhododendron ferrugineum stem cell extract. In a specific embodiment, the amount of plant stem cell extracts of the cosmetic composition is between 0.003 and 0.015 wt/wt %, preferably between 0.005 and 0.01 wt/wt %; the amount of the additional peptide composition is between 1·10⁻⁵ and 1·10⁻⁴ wt/wt %, preferably between 5·10⁻⁵ and 9·10⁻⁵ wt/wt %; and the amount of the vitamin composition is between 0.01 and 0.1 wt/wt %, preferably between 0.04 and 0.08 wt/wt %.

As used herein, the at least one additional peptide composition refers to a peptide composition other than a PGA composition. Generally, it comprises one or more additional peptides selected from acetyl hexapeptide-8, palmitoyl pentapeptide-4, palmitoyl tripeptide-1, palmitoyl tetrapeptide-7, dipeptide-2, hesperidin methyl chalcone and/or dipeptide diaminobutyroyl benzylamide diacetate. As used herein, at least one vitamin composition comprises vitamin C, vitamin E and/or vitamin A or a suitable derivative of these vitamins. A suitable derivative of a vitamin can for example be a precursor of a vitamin or a masked vitamin, which can release the actual vitamin upon treatment under suitable conditions. A typical example is tocopheryl acetate, which is a synthetic vitamin E derivative and which can release vitamin E by cleavage of the acetate group. Another example is ascorbyl glucoside, which is a glycosylated derivative of vitamin C.

The present invention provides in a further aspect the use of the compositions of the invention as a new skin care treatment based on the synergistic effects of active substances of the composition comprising (i) at least one gamma-PGA composition, (ii) at least one plant stem cell extract, (iii) at least one additional peptide composition and (iv) at least one vitamin composition.

Gamma PGA and its salts provide a skin moisturizing effect due to the following complementary mechanisms: a) Gamma PGA is known to inhibit hyaluronidases and therefore both the epidermal hyaluronic acid as well as any hyaluronic acid or its derivatives, which may be present in the cosmetic composition are preserved; b) Gamma PGA is an excellent water store as it can efficiently bind water molecules and therefore prevent the loss of water from the skin. As such, it also increases the natural moisturizing factor, which describes a group of substances in the surface of the epidermis being capable of binding water. In particular, gamma PGA molecules with a high-molecular weight of 1 MDa to 5 MDa, can retain moisture for a long time, even if the surrounding is very dry. Gamma PGA molecules with a low molecular weight of 300 to 1500 Da, are able to penetrate the skin and therefore can act as a reagent for the epidermis from within and/or even further increase the HA content of the skin. It has been shown in in vitro tests, that low molecular weight gamma PGA mediates an improved gene expression of matrix proteins, such as filaggrin, involucirn and loricrin. These proteins are primarily cross-linked in the cells by the enzyme transglutaminase-1. As the process of cross-linking greatly improves the stability of the epidermis, a higher expression rate of these genes leads to an improved vitality of the skin. Additionally, low molecular weight gamma PGA can aid to increase transglutaminase-1 expression, thereby further increasing this effect. Further studies have shown that low molecular weight gamma PGA is able to suppress MMP-1 gene expression. This process slows down the degradation of matrix proteins, which play an important role in maintaining the elasticity and firmness of the skin. Additionally, in the presence of low molecular weight gamma PGA the levels of several natural moisture factor components such as pyrrolidone carboxylic acid and urocanic acid considerably increase by 35 to 50%.

Plant stem cell extracts have been demonstrated to significantly shallow wrinkles and crow's feet. The extracts further include active substances, which can slow down the loss of skin firmness and the aging process of the skin. Plant stem cell extracts have also been associated with the protection of skin stem cells.

Various peptides have been shown to have a skin conditioning effect. Acetyl hexapeptide-8 helps to reduce the depth of wrinkles, in particular facial wrinkles. Palmitoyl pentapeptide-4 is able to support the synthesis of collagen I, II and fibronectin, which are present in the dermal layer of the skin as structural molecules. Palmitoyl tripeptide-1 and 7 can activate the synthesis of extracellular matrix macromolecules and therefore provide an anti-aging effect. Dipeptide diaminobutyroyl benzylamide diacetate is an antagonist of the muscular nicotinic acetylcholine membrane's receptor and an excellent anti-wrinkle active compound.

Vitamins such as vitamins C and E are both powerful antioxidants, which help to reduce skin irritation and stimulate collagen synthesis. Furthermore, vitamin A can stimulate the skin cell growth.

In a further aspect the invention provides the compositions of the invention in form of a topical formulation, which includes all the commonly used forms such as a liquid or solution, a serum, a cream, a paste, an ointment, a gel, a foam, a lotion, a milk or a spray. The term “liquid” or “solution” refers to a homogeneous mixture wherein the molecules of the solute (or dissolved substance) are uniformly distributed among those of the solvent. Solvents that may be useful in the compositions of the present disclosure include water, as well as organic solvents, such as the alcohols. The term “cream” as used herein refers to a viscous liquid or semisolid emulsion of either the oil-in-water or water-in-oil type. The term “emulsion” refers to a colloid system in which both the dispersed phase and the dispersion medium are immiscible liquids where the dispersed liquid is distributed in small globules throughout the body of the dispersion medium liquid. Common types of emulsions are oil-in-water, where oil is the dispersed liquid and an aqueous solution, such as water, is the dispersion medium, and water-in-oil, where, conversely, an aqueous solution is the dispersed phase. Typical creams of the oil-in-water type include hand creams and foundation creams. Water-in-oil creams include cold creams and emollient creams. The term “paste” refers to pasty formulations, which are semisolid dosage form that contains one or more substances intended for topical application. The term “ointment” refers to a semi-solid preparation. Ointment bases include typically hydrocarbon bases (oleaginous), adsorption bases (anhydrous), emulsion bases (water and oil type), and water soluble bases. They are more moisturizing and more occlusive than creams, and may form a protective film over the skin. The occlusive effect may be desired to prolong and enhance penetration. The term “gel” as used herein refers to a sticky, jelly-like semisolid or solid prepared from high molecular weight polymers in an aqueous or alcoholic base. Alcoholic gels may be desired to achieve a drying and/or cooling effect.

The compositions of the invention may be prepared using technology readily known in the cosmetic arts, such as those described in Remington: The Science and Practice of Pharmacy, 20th Ed. (Gennaro, A. R. et al., eds) Lippincott Williams and Wilkins: Philadelphia (2000), which is incorporated herein by reference. The preparation of suitable formulations based in the inventive compositions is also known in the art, e.g. Handbook of Cosmetic Science and Technology, Second Edition, Marc Paye, et al. (Editor), CRC Press (2006), which is incorporated herein by reference.

The compositions and formulations of the invention further comprise a carrier, preferably an aqueous carrier for providing the continuous phase. The carrier is contained in the compositions, for example, about 30 wt/wt % to about 98 wt/wt %, about 50 wt/wt % to about 95 wt/wt %, or about 70 wt/wt % to about 95 wt/wt %. Exemplary carriers include water and water solutions of lower alkyl alcohols. Exemplary lower alkyl alcohols include monohydric alcohols having 1 to 6 carbons, e.g., ethanol. According to some embodiments, the aqueous carrier is substantially water. The pH of the described compositions are, for example, about 4 to about 8, and may be adjusted as desired by including buffers and/or other pH adjusting agents can be included to achieve the desirable pH. Exemplary pH adjusters herein include acetates, phosphates, citrates, triethanolamines and carbonates.

Depending on the desired formulation, additional ingredients can be added to the inventive compositions, including, but not limited to, surfactants, emulsifying agents, emollients, preservatives, humectants, buffers, thickeners, UV protectants, provided that the selected additional component(s) is chemically and physically compatible (i.e. having no interaction that would substantially reduce the efficacy of the compositions under ordinary use conditions).

Surfactants include nonionic surfactants, e.g., polysorbates, polyoxyalkylene hydrogenated caster oils, polyglycerin alkyl esters having the C10-20 of alkyl substitute, polyoxyethylene sterols, and polyoxyethylene hydrogenated sterols. Exemplary polyoxyalkylene hydrogenated castor oils include polyoxyethylene hydrogenated castor oils having 20-100 moles of ethylene oxides, such as polyoxyethylene (20) hydrogenated castor oil, polyethylene (40) hydrogenated castor oil, and polyoxyethylene (100) hydrogenated castor oil. Exemplary polyglycerin alkyl esters include those having 6-10 moles of glycerin units, such as polyglyceryl-6 laurate, polyglyceryl-10 laurate, and polyglyceryl-10 stearate. Exemplary polysorbates include those having 20-80 moles of ethylene oxides, such as polysorbate-20, polyborbate-40, polysorbate-60, and polysorbate-80. Exemplary polyethylene sterols and polyethylene hydrogenated sterols include those having 10-30 moles of ethylene oxides, such as polyethylene (10) phytosterol, polyethylene (30) phytosterol, and polyethylene (20) cholesterol Hydrophobic nonionic surfactants (i.e. water insoluble surfactants) can be included as oily components. Exemplary hydrophobic nonionic surfactants include cetearyl glucoside, steareth-2, laureth-4, sucrose cocate, sorbitan monoisostearate, sorbitan diisostearate, sorbitan sesquiisostearate, sorbitan monooleate, sorbitan dioleate, sorbitan sesquioleate, glyceryl monoisostearate, glyceryl diiostearate, glyceryl sesquiisostearate, glyceryl monooleate, glyceryl dioleate, glyceryl sesquioleate, diglyceryl diisostearate, diglyceryl dioleate, diglycerin monoisostearyl ether, diglycerin diisostearyl ether, and mixtures thereof. Oily components may also include fatty alcohols, such as saturated, linear or branched C2-30 fatty alcohols, saturated, linear or branched C2-30 diols, and mixtures thereof. Further examples include cetyl alcohol, steacyl alcohol, and mixtures thereof.

Exemplary emollients useful in the cosmetic and dermatologic compositions disclosed herein include, but are not limited to, wood alcohols, fatty alcohols (e.g., cetyl alcohol), propylene glycol, cocamidopropyl betaine, butylene glycol, pentylene glycol, ethylhexylglycerine, methoxy PEG-17, PEG-22/dodecyl glycol copolymers, alkylglucosides; butters, such as aloe butter, almond butter, avocado butter, cocoa butter, coffee butter, hemp seed butter, kokum butter, mango butter, olive butter, sal butter, shea butter, glycerin, and oils, such as almond oil, aloe vera oil, apricot kernel oil, avocado oil, babassu oil, black cumin seed oil, borage seed oil, brazil nut oil, camellia oil, castor oil, coconut oil, emu oil, evening primrose seed oil, flaxseed oil, grape seed oil, hazelnut oil, hemp seed oil, jojoba oil, kukui nut oil, macadamia nut oil, meadowfoam seed oil, mineral oil, neem seed oil, olive oil, palm oil, palm kernel oil, peach kernel oil, peanut oil, plum kernel oil, pomegranate seed oil, poppy seed oil, pumpkin seed oil, rice bran oil, rosehip seed oil, safflower oil, sea buckthorn oil, sesame seed oil, shea nut oil, soybean oil, sunflower oil, tamanu oil, turkey red oil, walnut oil, or wheatgerm oil.

Exemplary emulsifiers include, without limitation, non-ionic and anionic emulsifiers, such as sugar esters and polyesters, alkoxylated sugar esters and polyesters, C1-30 fatty acid esters of C1-30 fatty alcohols (or alkoxylated derivatives thereof), alkoxylated ethers of C C1-30 fatty alcohols, polyglyceryl esters of C1-30 fatty acids, C1-30 esters or C1-30 ethers of polyols, alkyl phosphates, polyoxyalkylene fatty ether phosphates, fatty acid amides, acyl lactylates, soaps, and mixtures thereof; polyethylene glycol 20 sorbitan monolaurate (polysorbate 20), polyethylene glycol 5 soya sterol, steareth-20, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, polyoxyethylene 20 sorbitan trioleate (polysorbate 85), sorbitan monolaurate, polyoxyethylene 4 lauryl ether sodium stearate, polyglyceryl-4 isostearate, hexyl laurate, PPG-2 methyl glucose ether distearate, ceteth-10, diethanolamine cetyl phosphate, glyceryl stearate, PEG 40 hydrogenated castor oil, PEG-60 hydrogenated castor oil, and mixtures thereof.

Exemplary preservatives (i.e. compounds preventing the growth of undesired microorganisms in aqueous products) include, without limitation: ascorbic acid, ascorbyl palmitate, biopein, BHT (butylated hydroxyl-toluene), butylated hydroxyanisole, butylated hydroxytoluene, butylparaben, calcium ascorbate, calcium sorbate, citric acid, cinnamon cassia, chlorocresol, diazolidinyl urea, dilauryl thiodipropionate, EDTA (ethylenediamine tetraacetic acid tetrasodium salt), erythorbic acid, grapefruit seed extract, hydroxyhenzoates, methylparaben, Neopein, phenonip, phenoxyethanol, potassium bisulfite, potassium metabisulfite, potassium sorbate, propylparaben, rosemary oil extract, sodium ascorbate, sodium benzoate, sodium bisulfite, sodium metabisulfite, sodium sorbate, sodium sulfite, sorbic acid, sulfur dioxide, Suprarein, thiodipropionic acid, and/or tocopherols.

Exemplary water-soluble humectants (i.e. compounds promoting water retention due to their hygroscopicity) include, without limitation, polyhydric alcohols, such as butylene glycol (1,3 butanediol), pentylene glycol (1,2-pentanediol), glycerin, sorbitol, propylene glycol, hexylene glycol, ethoxylated glucose, 1,2-hexane diol, 1,2-pentane diol, hexanetriol, dipropylene glycol, erythritol, trehalose, diglycerin, xylitol, maltitol, maltose, glucose, fructose; and other water-soluble compounds such as urea, sodium chondroitin sulfate, sodium hyaluronate, sodium adenosin phosphate, sodium lactate, pyrrolidone carbonate, glucosamine, cyclodextrin, and mixtures thereof. Additional examples include water soluble alkoxylated nonionic polymers such as polyethylene glycols and polypropylene glycols of molecular weight of up to about 1000 (e.g., PEG-200, PEG-400, PEG-600, PEG-1000), and mixtures thereof. Commercially available humectants include, without limitation: butylene glycol (1,3-Butylene glycol, Celanese), pentylene glycol (HYDROLITE™-5 Dragoco), glycerin (STAR™ and SUPEROL™, The Procter and Gamble Company, CRODEROL™ GA7000 Croda Universal Ltd., PRECERIN™ series Unichema, NOF; propylene glycol (LEXOL™ PG-865/855, Inolex, 1,2-PROPYLENE GLYCOL USP, BASF; sorbitol (LIPONIC™ series, Lipo, SORBO™, ALEX™, A-625™, and A-641™, ICI, and UNISWEET™ 70, UNISWEET™ CONC, UPI; diglycerin (DIGLYCEROL™, Solvay GmbH); xylitol, Kyowa and Eizai; maltitol (MALBIT™, Hayashibara; sodium chondroitin sulfate, Freeman and Bioiberica; sodium hyaluronate, Chisso Corp. AVIAN SODIUM HYALURONATE series, Intergen, HYALURONIC ACID Na, lchimaru Pharcos; sodium adenosine phosphate, Daiichi Seiyaku; sodium lactate, Merck, etc, cyclodextrin (CAVITRON™, American Maize, RHODOCAP™ series, Rhone-Poulenc, and DEXPEARL™, Tomen); polyethylene glycols (CARBOWAX™ series, Union Carbide), and a mixture of glyceryl polymethacrylate, propylene glycol and PVM/MA copolymer (LUBRAJEL™ Oil, Guardian Lab).

Exemplary polymeric thickeners in the compositions of the invention include, for example, casein, gelatin, starch, gum, pectin, phycocolloids, and synthetic polymers. Examples of the abovementioned materials are salts of alginic acid and derivatives thereof, including, for example, sodium alginate and propylene glycol alginate, acacia gum, carrageen, guar gum, karaya gum, locust bean gum, tragacanth gum, xanthan gum, hyaluronic acid and salts thereof, such as, for example, sodium hyaluronate, gelatin and polydextrose. Other polymeric thickeners, which may be used, include, for example, acrylic acid polymers, such as crosslinked acrylic acid interpolymers, polyacrylic acid and salts thereof, crosslinked acrylic acid homopolymers, crosslinked acrylic acid copolymers, acrylic/acrylate copolymers which are dimethicone copolyols, polyacrylamide, poly[(sodium acrylate)-co-(vinyl alcohol)], sodium polymethacrylate, sodium polystyrene sulfonate, poly(ethylene-co-sodium acrylate), poly(acrylamide-co-sodium acrylate), povidone and derivatives thereof, polyquaternium compounds, such as polyquaternium 10, polyvinyl alcohol, polyethylene oxide and poloxamers. Other thickeners may be acrylic acid copolymers, with the crosslinked acrylic acid copolymers being more preferred. Particularly preferred among these thickeners are acrylate/C10-30 alkyl acrylate crosspolymers. Examples of acrylate/C10-30 alkyl acrylate crosspolymers, which may be suitable for use in the compositions of the invention, are polymeric emulsifiers Pemulen®, including Pemulen® TR1 and Pemulen® TR2. Alternatively, the compositions may include inorganic thickeners. Suitable inorganic thickeners include, for example, bentonite, magnesium aluminum silicate and colloidal silica. Exemplary commercially available compounds include, without limitation, xanthan gum (KELTROL™, Kelco), Carbomers (CARBOPOL™ 934, CARBOPOL™ 940, CARBOPOL™ 950, CARBOPOL™ 980, and CARBOPOL™ 981 (B. F. Goodrich Company), acrylates/steareth-20 methacrylate copolymer (ACRYSOL™ 22 (Rohm and Hass), polyacrylamide (SEPIGEL™ 305, Seppic), glyceryl polymethacrylate (LUBRAGEL™ NP, LUBRAGEL™ OIL, ISP), scleroglucan (CLEAROGEL™ SCI I, Michel Mercier Products Inc.), ethylene oxide and/or propylene oxide based polymers (CARBOWAX™ PEGs, POLYOX™ WASRs, and UCON™ FLUIDS (Amerchol). Other exemplary agents include commercially available amphoteric polymers such as Polyquaternium 22 (MERQUAT™ 280, MERQUAT™ 295), Polyquaternium 39 (MERQUAT™ PLUS 3330, MERQUAT™ PLUS 3331), and Polyquaternium 47 (MERQUAT™ 2001, MERQUAT™ 200 IN) (Calgon Corporation).

Exemplary UV protecting agents include, without limitation, 2-ethylhexyl-p-methoxycinnamate (commercially available as PARSOL™ MCX), butylmethoxydibenzoyl-methane, 2-hydroxy-4-methoxybenzo-phenone, 2-phenylbenzimidazole-5-sulfonic acid, octyldimethyl-p-aminobenzoic acid, octocrylene, 2-ethylhexyl N,N-dimethyl-p-aminobenzoate, p-aminobenzoic acid, 2-phenylbenzimidazole-5-sulfon c acid, octocrylene, oxybenzone, homomenthyl salicylate, octyl salicylate, 4,4′-methoxy-t-butyldibenzoylmethane, 4-isopropyl dibenzoylmethane, 3-benzylidene camphor, 3-{4-methylbenzylidene) camphor, EUSOLEX™ 6300, Octocrylene, Avobenzone (commercially available as PARSOL 1789), and mixtures thereof.

The following examples are only to be regarded as representative examples and should by no means be understood as a restriction of the scope of the present invention. From the disclosure as a whole, the skilled person is presented with different modifications and alternative embodiments, which are within the scope of the claims.

### EXEMPLARY EMBODIMENTS

### Example 1: Experimental Procedure

In a first step deionized water is heated to 80° C. in the vessel. Hydrolyzed PGA is added under stirring at 80° C. until completely dissolved. Subsequently the mixture is cooled to 50° C. and the stem cells in form of a granulate are added to the solution under stirring at 50° C. until full dissolution is obtained. Subsequently the mixture is cooled to 40° C. Preservatives of choice are added under stirring until they are completely dispersed and the mixture is cooled to room temperature. In a further step the peptide composition (preferably in form of a solution) is added to the vessel under stirring until completely dissolved. The obtained solution is allowed to rest for 24 h, and subsequently if necessary subjected to filtration for purification.

An exemplary composition of the invention comprises the following components:

| | | wt/ |
|---|---|---|
| Ingredient | CAS No. | wt % |
| Gamma PGA composition | | |
| Potassium hydrolyzed | 63950-69-0 | 0.1 |
| polygamma-glutamate | | |
| Vitamin composition | | |
| Ascorbyl glucoside | 129499-78-1 | 0.05 |
| Tocopheryl acetate | 7695-91-2 | 0.01 |
| Stem cell composition | | |
| Malus Domestica fruit cell | 89957-48-2 | 0.009 |
| culture extract | | |
| Argania Spinosa callus cell | 1083089-29-5 | 0.00016 |
| culture extract | | |
| Rhododendron Ferrugineum | 90106-21-1 | 0.00016 |
| leaf cell culture extract | | |
| Peptide composition | | |
| Acetyl hexapeptide-8 | 616201-22-9 | 0.00005 |
| Palmitoyl pentapeptide-4 | 214047-00-4 | 0.000015 |
| Palmitoyl tripeptide-1 | 147732-56-7 | 0.000015 |
| Palmitoyl tripeptide-7 | 221227-05-0 | 0.000075 |
| Additives | | |
| Glycerin | 56-81-5 | 8.42 |
| Propylene glycol | 57-55-6 | 4.0 |
| Polysorbate 20 | 9005-64-5 | 1.5 |
| VP/VA Copolymer | 25086-89-9 | 1.45 |
| Hydroxyethylcellulose | 9004-62-0 | 1 |
| Phenoxyethanol | 122-99-6 | 0.89 |
| Glyceryl acrylate/acrylic | 9003-01-4 | 0.36 |
| acid copolymer | | |
| Ethylhexylglycerin | 70445-33-9 | 0.3 |
| Chlorphenesin | 104-29-0 | 0.25 |
| Disodium EDTA | 139-33-3 | 0.1 |
| Limonene | 5989-27-5 | 0.089 |
| Imperate cylindrical root | 999999-99-4 | 0.08 |
| extract | | |
| Butylene glycol | 107-88-0 | 0.06 |
| Maltodextrin | 9050-36-6 | 0.046 |
| Pentadecalactone | 106-02-5 | 0.045 |
| Isomalt | 64519-82-0 | 0.0372 |
| Pullulan | 9057-02-7 | 0.036 |
| Glycyrrhetinic acid | 471-53-4 | 0.03 |
| Caramel | 8028-89-5 | 0.0192 |
| Leontopodium alpinum | 391900-47-3 | 0.016 |
| extract | | |
| Camellia sinensis leaf | 84650-60-2 | 0.015 |
| extract | | |
| Citrus medica limonum | 8008-56-8 | 0.011 |
| (lemon) peel oil | | |
| PEG-8 | 5117-19-1 | 0.01 |
| Sodium hyaluronate | 9067-32-7 | 0.01 |
| Hydrolyzed rice protein | 94350-05-7 | 0.008 |
| Macrocystis pyrifera (kelp) | 347174-92-9 | 0.008 |
| extract | | |
| Centella asiatica extract | 84696-21-9 | 0.006 |
| Echinacea angustifolia root | 84696-11-7 | 0.006 |
| extract | | |
| Carbomer | 9003-01-4 | 0.005 |
| Nannochloropsis oculata | 92128-82-0 | 0.005 |
| extract | | |
| Poly(vinylpolypyrrolidinone) | 9003-39-8 | 0.004 |
| Glycine soy (sojybean) | 9010-10-0 | 0.003 |
| protein | | |
| Superoxide dismutase | 9054-89-1 | 0.003 |
| Citrus aurantium dulcis | 8008-57-9 | 0.0025 |
| (orange) peel oil | | |
| Citral | 5392-40-5 | 0.0021 |
| Hydrolyzed vegetable | 100209-45-8 | 0.002 |
| protein | | |
| Xanthan gum | 11138-66-2 | 0.001 |
| Lecithin | 8002-43-5 | 0.00054 |
| Caprylyl glycol | 1117-86-8 | 0.00005 |
| Sodium benzoate | 1362738-67-7 | 0.0001 |
| Coenochloris signiensis | 532-32-1 | 0.00006 |
| extract | | |
| Carrier | | |
| Water | 7732-18-5 | 81 |

1. A cosmetic composition comprising:
(i) at least one gamma-PGA composition;
(ii) at least one plant stem cell extract;
(iii) at least one additional peptide composition; and
(iv) at least one vitamin composition.

2. The cosmetic composition according to claim 1, wherein the gamma-PGA composition comprises gamma-PGA and/or an acceptable salt thereof, preferably potassium hydrolyzed polygamma-glutamate.

3. The cosmetic composition according to claim 1, wherein the gamma-PGA composition comprises at least one of an amount of low-molecular weight gamma-PGA, or acceptable salt thereof with a molecular weight between 300 and 1500 Da, preferably between 800 and 1200 Da, and an amount of high-molecular weight gamma-PGA or acceptable salt thereof with a molecular weight between 1 MDa and 5 MDa, preferably between 2 MDa and 3 MDa.

4. The cosmetic composition according to claim 1, wherein the at least one plant cell extract is selected from one or more of apple stem cell extract, preferably Malus domestica stem cell extract; argan stem cell extract, preferably Argania spinosa stem cell extract and alp rose stem cell extract, preferably Rhododendron ferrugineum stem cell extract.

5. The cosmetic composition according to claim 1, wherein the additional peptide composition comprises one or more of acetyl hexapeptide-8, palmitoyl pentapeptide-4, palmitoyl tripeptide-1, palmitoyl tetrapeptide-7, dipeptide-2, hesperidin methyl chalcone and dipeptide diaminobutyroyl benzylamide diacetate.

6. The cosmetic composition according to claim 1, wherein the vitamin composition comprises vitamin C, vitamin E and/or vitamin A or a suitable derivative of these vitamins.

7. The cosmetic composition according to claim 1, wherein the amount of the gamma-PGA composition is between 0.05 and 4 wt/wt %, preferably between 0.1 and 1 wt/wt %.

8. The cosmetic composition according to claim 1, wherein the amount of the at least one plant stem cell extract is between 0.003 and 0.015 wt/wt %, preferably between 0.005 and 0.01 wt/wt %; the amount of the additional peptide composition is between 1·10⁻⁵ and 1·10⁻⁴ wt/wt %, preferably between 5·10⁻⁵ and 9·10⁻⁵ wt/wt %; and the amount of the vitamin composition is between 0.01 and 0.1 wt/wt %, preferably between 0.04 and 0.08 wt/wt %.

9. The cosmetic composition according to claim 1, wherein the gamma-PGA composition comprises potassium hydrolyzed polygamma-glutamate; the cosmetic composition further comprises apple stem cell extracts, argan stem cell extracts and alp rose stem cell extracts; the additional peptide composition comprises acetyl hexapeptide-8, palmitoyl pentapeptide-4, palmitoyl tripeptide-1 and palmitoyl tetrapeptide-7; and the vitamin composition comprises ascorbyl glucoside and tocopheryl acetate.

10. The cosmetic composition according to claim 1, wherein the cosmetic composition further comprises hyaluronic acid or a suitable salt thereof, such as sodium or potassium hyaluronate, preferably the amount of hyaluronic acid or a suitable salt thereof is between 0.005 and 0.03 wt/wt %.

11. The cosmetic composition according to claim 1, wherein the cosmetic composition further comprises superoxide dismutase and/or glycine soy protein, preferably the amount of the superoxide dismutase and/or glycine soy protein is each in between 0.001 and 0.01 wt/wt %.

12. The cosmetic composition according to claim 1, wherein the cosmetic composition is in the form of a serum, a crème or a foam.

13. A cosmetic method for increasing the natural moisturizing factor of the skin and/or for prevention of premature skin degradation and/or for the prevention of age spots comprising the step of topically applying a cosmetic composition according to claim 1 to the human skin.
